(19) United States
(12) Patent Application Publication
(10) Pub. No.: US 2003/0118989 A1
(43) Pub. Date: Jun. 26, 2003

(54) Differential isolation of cellular content for biological assays

(76) Inventors: Carsten Rosenow, Redwood City, CA (US); Rini Mukherjee Saxena, Mountain View, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(21) Appl. No.: 09/683,405

(22) Filed: Dec. 21, 2001

(51) Int. Cl. C12Q 1/68; C12N 1/08

(52) U.S. Cl. 435/6; 435/270

(57) ABSTRACT

In one embodiment, methods are provided for differential isolation of cellular contents from different cells for further analysis. In one exemplary embodiment, the nucleic acids of bacteria are isolated from a mixture of bacteria and animal cells by lysing animal cells.

### BACKGROUND OF INVENTION

The present invention is in the field of genetic analysis for medical diagnosis, genetic variation research, or genetic engineering. More specifically, the present invention is in the field of nucleic acid analysis.

During the course of a human infection caused by pathogenic bacteria, eukaryotic and prokaryotic cells go through a cell-cell interaction process that determines the outcome of the infection. Understanding the progress of this interaction is the first step for a successful therapy and can provide new targets for microbial drug discovery efforts.

### SUMMARY OF INVENTION

In one aspect of the invention, methods are provided for differential isolation of cellular contents from interested cells in a sample with mixed cell population. Some embodiments of the invention employ differential lysis of cells to isolate contents of different cells (e.g., bacteria cells vs. animal cells) for further analyses such as gene expression monitoring (including protein assays) or microorgnism genetic identification using oligonucleotide probe arrays.

In some embodiments, the methods include obtaining a sample comprising a first type of cells and a second type of cells, where the first type of cells is at least twice as susceptible to a lysis agent as the second type of cells; and applying the lysis agent to break the first type of cells. As used herein, the term lysis agent refers to any physical, biological, or chemical force that causes the break down of cell walls or cell membranes. Exemplary lysis agents include detegents, enzymes, sonication, heat, mechanic forces, etc.

In some embodiments, the first type of cells is at least 5 or 10 times more susceptible to the lysis agent than the second type of cells. In some embodiments, at least 60%, 80%, 90% or 99% of the second type of cells are removed by, for example, centrifugation.

The first type of cells can be animal cells and the second type of cells are bacteria cells. In some other embodiments, the first type of cells are animal ??? cells and the second type of cells are plant cells or fungi (yeast) cells. In such embodiments, the lysis agent can be a detergent or incubation with a detergent.

The method can also be used to separate bacterial cells from different species. For example, the first type of cells can be gram negative bacteria and the second type of cells can be gram positive bacteria. The lysis agent can be a relatively mild lysosome digestion followed by a cell membrane lysis agent with the conditions that is sufficient for lysis of gram negative bacteria and not sufficient for lysing gram positive bacteria.

In another exemplary embodiment, the first type of cells are yeast cells and the second type of cells are bacteria or plant cells. The lysis agent is a zymolase, glucalase or lyticase digestion followed by a cell membrane lysis agent.

In another aspect of the invention, methods are provided for detecting nucleic acids. The methods include the steps of obtaining a sample containing a first type of cells and a second type of cells, where the first type of cells is at least twice as susceptible to a lysis agent as the second type of cells; applying the lysis agent to break the first type of cells; removing at least 60% of the second type of cells to obtain an isolate; preparing a nucleic acid sample from the isolate.

In some embodiments, the first type of cells is at least 5 or 10 times more susceptible to the lysis agent than the second type of cells. In some embodiments, at least 80%, 90% or 99% of the second type of cells are removed.

The nucleic acid sample may be used to hybridize to a plurality of, at least 100, 1000, 10000, 300,000 different nucleic acid probes. The probes may be immobilized on a substrate which can be solid surface such a glass plate or slide, a gel matrix. In such embodiments, each of the different probes is immobilized on an addressable location. The probes may also be immobilized, for example, on beads or on optical fibers.

The first type of cells can be animal (including human) cells and the second type of cells are bacteria cells. In some other embodiments, the first type of cells are animal cells and the second type of cells are plant cells or fungi cells. In such embodiments, the lysis agent can be a detergent or incubation with a detergent.

The method can also be used to separate bacterial cells from different species. For example, the first type of cells can be gram negative bacteria and the second type of cells can be gram positive bacteria. The lysis agent can be a relatively mild lysosome digestion followed by a cell membrane lysis agent with the conditions that is sufficient for lysis of gram negative bacteria and not sufficient for lysing gram positive bacteria.

The nucleic acid sample can be used for gene expression monitoring. In such embodiments, the nucleic acid sample contains RNA transcripts from the first type of cells or nucleic acids derived from the RNA transcripts from the first type of cells and wherein the nucleic acid probes are for detecting gene expression.

In another exemplary embodiment, genomic DNA from the first type of cells or nucleic acids derived from the genomic DNA from the first type of cells are hybridized with the nucleic acid probes that are designed for identifying the species of the first type of cells according to the genomic DNA sequence In yet another aspect of the invention, methods for detecting nucleic acids from the second type of cells are provided. In some embodiments, the methods include obtaining a sample comprising a first type of cells and a second type of cells, where the first type of cells is at least twice as susceptible to a lysis agent as the second type of cells; applying the lysis agent to break the first type of cells; removing at least 80% of the cellular content of first type of cells; lysing second type of cells to obtain an isolate; and preparing a nucleic acid sample from the isolate.

### BRIEF DESCRIPTION OF DRAWINGS

The accompanying drawings, which are incorporated in and form a part of this specification, illustrate embodiments of the invention and, together with the description, serve to explain the embodiments of the invention: FIG. 1A is a scanned image of RNAs from E. Coli cells on a high density oligonucleotide probe array.

FIG. 1B is a scanned image of RNAs hybridized with the same type of array of FIG. 1A. The RNA is isolated from a mixture of E. Coli cells and human cells by first lysing human cells with 0.2% triton.

FIG. 1C is a scanned image of RNAs from a mixture of E. Coli cells and human cells on a high density oligonucleotide probe array.

### DETAILED DESCRIPTION

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention.

### General

The present invention relies on many patents, applications and other references for certain details well known to those of the art. Therefore, when a patent, application, or other reference is cited or repeated below, it should be understood it is incorporated by reference in its entirety for all purposes as well as for the proposition that is recited.

As used in the specification and claims, the singular form a, an, and the include plural references unless the context clearly dictates otherwise. For example, the term an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from a 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques of organic chemistry, polymer technology, molecular biology (including recombinant techniques), cell biology, biochemistry, and immunology, which are within the skill of the art. Such conventional techniques include polymer array synthesis, hybridization, ligation, detection of hybridization using a label. Such conventional techniques can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), all of which are herein incorporated in their entirety by reference for all purposes.

Additional methods and techniques applicable to array synthesis have been described in U.S. Pat. Nos. 5,143,854, 5,242,974, 5,252,743, 5,324,633, 5,384,261, 5,405,783, 5,412,087, 5,424,186, 5,445,934, 5,451,683, 5,482,867, 5,489,678, 5,491,074, 5,510,270, 5,527,681, 5,550,215, 5,571,639, 5,578,832, 5,593,839, 5,599,695, 5,624,711, 5,631,734, 5,677,195, 5,744,101, 5,744,305, 5,770,456, 5,795,716, 5,800,992, 5,831,070, 5,837,832, 5,856,101, 5,871,928, 5,858,659, 5,936,324, 5,968,740, 5,974,164, 5,981,185, 5,981,956, 6,025,601, 6,033,860, 6,040,1 38, and 6,090,555, which are all incorporated herein by reference in their entirety for all purposes.

Analogue when used in conjunction with a biomonomer or a biopolymer refers to natural and un-natural variants of the particular biomonomer or biopolymer. For example, a nucleotide analogue includes inosine and dideoxynucleotides. A nucleic acid analogue includes peptide nucleic acids. The foregoing is not intended to be exhaustive but rather representative. More information can be found in U.S. Pat. No. 6,156,501.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% or 95%, and more preferably from about 98 to 100%. Alternatively, substantial complementarity exists when an RNA or DNA strand will hybridize under selective hybridization conditions to its complement. Typically, selective hybridization will occur when there is at least about 65% complementarity over a stretch of at least 14 to 25 nucleotides, preferably at least about 75%, more preferably at least about 90% complementarity. See e. g., M. Kanehisa Nucleic Acids Res. 12:203 (1984), incorporated herein by reference.

Hybridization refers to the process in which two single-stranded polynucleotides bind non-covalently to form a stable double-stranded polynucleotide; triple-stranded hybridization is also theoretically possible. The resulting (usually) double-stranded polynucleotide is a hybrid. The proportion of the population of polynucleotides that forms stable hybrids is referred to herein as the degree of hybridization. Hybridizations are usually performed under stringent conditions, for example, at a concentration of no more than 1 M and a temperature of at least 25° C. For example, conditions of 5×SSPE (750NaCl, 50NaPhosphate, 5EDTA, pH 7.4) and a temperature of 25-30° C. are suitable for allele-specific probe hybridizations. For stringent conditions, see, for example, Sambrook, Fritsche and Maniatis. Molecular Cloning A laboratory Manual 2^{nd} Ed. Cold Spring Harbor Press (1989) which is hereby incorporated by reference in its entirety for all purposes above.

Nucleic acid refers to a polymeric form of nucleotides of any length, such as oligonucleotides or polynucleotides, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be customized to stabilize or destabilize hybrid formation or enhance the specificity of hybridization with a complementary nucleic acid sequence as desired.

Oligonucleotide or polynucleotide is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). The invention also encompasses situations in which there is a nontraditional base pairing such as Hoogsteen base pairing which has been identified in certain tRNA molecules and postulated to exist in a triple helix. Polynucleotide and oligonucleotide are used interchangeably in this application.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

Primer is a single-stranded oligonucleotide capable of acting as a point of initiation for template-directed DNA synthesis under suitable conditions, e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 3 to 6 and up to 30 or 50 nucleotides. Short primer molecules generally require cooler temperatures to form sufficiently stable hybrid complexes with the template. A primer needs not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Substrate refers to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

High density nucleic acid probe arrays, also referred to as DNA Microarrays, have become a method of choice for monitoring the expression of a large number of genes.

A target molecule refers to a biological molecule of interest. The biological molecule of interest can be a ligand, receptor, peptide, nucleic acid (oligonucleotide or polynucleotide of RNA or DNA), or any other of the biological molecules listed in U.S. Pat. No. 5,445,934 at col. 5, line 66 to col. 7, line 51. For example, if transcripts of genes are the interest of an experiment, the target molecules would be the transcripts. Other examples include protein fragments, small molecules, etc. Target nucleic acid refers to a nucleic acid (often derived from a biological sample) of interest. Frequently, a target molecule is detected using one or more probes. As used herein, a probe is a molecule for detecting a target molecule. It can be any of the molecules in the same classes as the target referred to above. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to targets or probes as nucleic acids, it should be understood that there are illustrative embodiments that are not to limit the invention in any way.

In preferred embodiments, probes may be immobilized on substrates to create an array. An array may comprise a solid support with peptide or nucleic acid or other molecular probes attached to the support. Arrays typically comprise a plurality of different nucleic acids or peptide probes that are coupled to a surface of a substrate different, known locations. These arrays, also described as “microarrays” or colloquially “chips” have been generally described in the art, for example, in Fodor et al., Science, 251:767-777 (1991), which is incorporated by reference for all purposes. Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668, U.S. Pat. Nos. 5,677,195, 5,800,992 and 6,156,501 which disclose methods of forming vast arrays of peptides, oligonucleotides and other molecules using, for example, light-directed synthesis techniques. See also, Fodor et al., Science, 251, 767-77 (1991). These procedures for synthesis of polymer arrays are now referred to as VLSIPS™ procedures. Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743, 6,140,044 and D430024, all of which are incorporated by reference in their entireties for all purposes.

Methods for signal detection and processing of intensity data are additionally disclosed in, for example, U.S. Pat. Nos. 5,547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. Nos. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174, 5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996, 6,136,269 and attorney docket numbers 3298.1 and 3309, all of which are incorporated by reference in their entireties for all purposes.

The embodiments of the invention will be described using GeneChip® high oligonucleotide density probe arrays (available from Affymetrix, Inc., Santa Clara, Calif., USA) as exemplary embodiments. One of skill the art would appreciate that the embodiments of the invention are not limited to high density oligonucleotide probe arrays. In contrast, the embodiments of the invention are useful for analyzing any parallel large scale biological analysis, such as those using nucleic acid probe array, protein arrays, etc.

Gene expression monitoring using GeneChip® high density oligonucleotide probe arrays are described in, for example, Lockhart et al., 1996, Expression Monitoring By Hybridization to High Density Oligonucleotide Arrays, Nature Biotechnology 14:1675-1680; U.S. Pat. Nos. 6,040,138 and 5,800,992, all incorporated herein by reference in their entireties for all purposes.

### Differential Isolation of Cellular Content

In one aspect of the invention, methods are provided for detecting biological molecules from one species in a mixed cell population with cells from two or more species. Some embodiments of the invention employs differential lysis of cells to isolate contents of different cells (e.g., bacteria cells vs. animal cells) for further analyses such as gene expression monitoring or microorgnism genetic identification using oligonucleotide probe arrays.

The methods of invention have extensive applications in fields such as plant pathology, infectious disease, biological warfare agent detection, food inspection, etc. For example, the gene expression of infecting pathogens and a plant can be studied by separately isolating the bacterial RNA and plant RNA. Similarly, the infection process in an animal tissue can be monitored by detecting the expression of bacterial gene expression using bacterial RNA isolated from a tissue sample that contains cells from the host. The methods are particularly useful for detecting and identifying biological agents present in a tissue sample. In such embodiments, the bacterial DNA can be isolated without or with the minimal contamination of DNA from host tissue. Similarly, bacterial contaimination of foods may be better detected by isolating bacterial nucleic acids from plant or animal nucleic acids normally present in many types of foods.

The differential lysis may be dependent upon the chemical, physical and other properties of the cell walls (or the lack of it) and membrane in various cells. For example, differential lysis can be achieved between animal cells and bacterial cells, plant cells and yeast cells, because animal cells lack cell walls. Differential lysis may also be achieved among bacterial cells because of the different chemical and physical structure of bacterial cell walls in different species (e.g., Gram negative vs. Gram positive).

The bacterial cell wall is a unique structure which surrounds the cell membrane. (see, e.g., Zinsser Microgiology, Zinsser, 20th Ed., 1992, Norwalk, Conn., Appleton & Lange, which is incorporated herein by reference). Although it is not present in every bacterial species, the cell wall is present in most bacteria. However, cell wall contents in bacteria are not identical. In fact, cell wall composition is one of the most important factors in bacterial species analysis and differentiation.

There are two major types of walls: Gram-positive and Gram-negative. The cell wall of Gram-positive bacteria consists of many polymer layers of peptidoglycan connected by amino acid bridges. The peptidoglycan polymer is composed of an alternating sequence of N-acetylglucosamine and N-acetyl-muraminic acid. Each peptidoglycan layer is connected, or crosslinked, to the other by a bridge made of amino acids and amino acid derivatives. The particular amino acids vary among different species, however. The crosslinked peptidoglycan molecules form a network which covers the cell like a grid. Also, 90% of the Gram-positive cell wall is comprised of peptidoglycan.

The cell wall of Gram-negative bacteria is much thinner, being comprised of only 20% peptidoglycan. Gram-negative bacteria also have two unique regions which surround the outer plasma membrane: the periplasmic space and the lipopolysaccharide layer. The periplasmic space separates the outer plasma membrane from the peptidoglycan layer. It contains proteins which destroy potentially dangerous foreign matter present in this space. The lipopolysaccharide layer is located adjacent to the exterior peptidoglycan layer. It is a phospholipid bilayer construction similar to that in the cell membrane and is attached to the peptidoglycan by lipoproteins. The lipid portion of the LPS contains a toxic substance, called Lipid A, which is responsible for most of the pathogenic affects associated with harmful Gram-negative bacteria. Polysaccharides which extend out from the bilayer also contibute to the toxicity of the LPS. The LPS, lipoproteins, and the associated polysaccharides together form what is known as the outer membrane.

Plant cell walls are distinguished from animal cells by the presence, around the plasmalemma, of a wall within which complex physicochemical and enzymatic phenomena progress. In the course of cell growth the dimensions of the cell wall vary according to the type of macromolecule of which it is composed. The first wall deposited after cell division is called the middle lamella and is essentially composed pectic material. The cell then lays down a wall composed of pecto-cellulosic material to supplant the middle lamella-the “primary” cell wall.

In fact the primary cell wall is a glycoproteinaceous layer composed of pectin, cellulose, hemicellulose and proteins. As the cell ages and differentiates it secretes new materials which form a mixture with the constituents of the primary cell wall so leading to the formation of a “secondary” cell wall. The nature of the constituents of the secondary cell wall depends on the cell type and the tissue to which the cell belongs. In general totally differentiated cells have stopped expanding and cannot divide further.

Young plant cell walls represent a structure which is simultaneously rigid and dynamic. Indeed, rigidity is required to counterbalance the effect of turgor pressure on the plasmalemma. To allow cell extension to occur the cell wall structure must be deformable. This dual functionality of cell walls is achieved through the mixture of polysaccharides and proteins.

Cellulose chains are formed into microfibrils which constitute the basic framework of the cell conveying a great resistance to tensile forces. The cellulose microfibrils represent about 20-30% of dry weight cell wall material occupying about 15% of cell wall volume. In cell walls that have differentiated and synthesised a secondary cell, the proportion of cellulose reaches 40-90% of the wall biomass. In the final stages of cell wall differentiation, notably in the middle lamella and primary cell wall, other wall polymers “lignins” are incorporated into the spaces around the polysaccharide fibrillar elements to form lignin-polysaccharides. Lignins arise from free-radical polymerisation of alcohols of para-hydroxy cinnamic acid and constitute between 10-30% of dry weight of wood, placing them second to cellulose. They contribute to the mechanical strength of the plant cell wall and confer resistance to pathogens.

The hemicelluloses constitute a large number of different polysaccharide molecules actually form a matrix for the cellulose microfibrils involving molecular interactions such as hydrogen bonds and van der Waal's forces.

Pectins constitute a major component of dicotyledon higher plants, about 35% of dry weight cell wall. In monocotyledons their proportion is less and their type is different.

The plant cell wall contains range of proteins which are implicated in the organisation and metabolism of the cell wall. The structural proteins can be gathered into five main families: extensins (rich in hydroxyproline), proteins rich in glycine (GRP), proteins rich in proline (PRP); lectins, proteins associated with arabinogalactans (AGP).

The rigid cell wall of fungi is a stratified structure consisting of chitinous microfibrils embedded in a matrix of small polysaccharides, proteins, lipids, inorganic salts, and pigments that provides skeletal support and shape to the enclosed protoplast. Chitin is a (b1-4)-linked polymer of N-acetyl-D-glucosamine (GIcNAc).

The major polysaccharides of the fungi cell wall matrix consist of noncellulosic glucans such as glycogen-like compounds, mannans (polymers of mannose), chitosan (polymers of glucosamine), and galactans (polymers of galactose). Small amounts of fucose, rhamnose, xylose, and uronic acids may be present.

Many fungi, especially the yeasts, have soluble peptidomannans as a component of their outer cell wall in a matrix of a- and b-glucans. Mannans, galactomannans, and, less frequently, rhamnomannans are responsible for the immunologic response to the medically important yeasts and molds. Mannans are polymers of mannose or heteroglucans with a-D-mannan backbones. Structurally, mannan consists of an inner core, outer chain, and base-labile oligomannosides.

In addition to chitin, glucan, and mannan, fungi cell walls may contain lipid, protein, chitosan, acid phosphatase, a-amylase, protease, melanin, and inorganic ions such as phosphorus, calcium, and magnesium. The outer cell wall of dermatophytes contains glycopeptides that may evoke both immediate and delayed cutaneous hypersensitivity. In the yeast Candida albicans, for example, the cell wall contains approximately 30 to 60 percent glucan, 25 to 50 percent mannan (mannoprotein), 1 to 2 percent chitin (located primarily at the bud scars in the parent yeast cell wall), 2 to 14 percent lipid, and 5 to 15 percent protein. The proportions of these components vary greatly from fungus to fungus.

Varying structure of the cells in plants, bacteria and fungus provides a differential property for the methods of the invention to differentially lysis cells and subsequently isolate differential cell contents for further analysis. One of skill in the art would appreciate that the differentially isolated cell contents can be used for many types of analysis includeing nucleic acids and protein assays. In one embodiment, the differentially isolated proteins are assayed using a protein array.

Physical, enzymatic and chemical forces or their combination can be used to break the cell walls and cell membranes.

Mechanical methods for disrupting fresh tissue and cells include homogenization with a Dounce or with a mechanical homogenizer (such as the Brinkmann Polytron”), vortexing, sonication, French press, bead milling, and even grinding in a coffee grinder.

Lysozyme and zymolase digestion are among the enzymatic methods frequently used with bacteria and yeast to dissolve a coat, capsule, capsid or other structure not easily sheared by mechanical methods alone. Enzymatic treatment is usually followed by sonication, homogenization or vigorous vortexing in a GITC lysis buffer for RNA isolation. Enzymatic methods may also be used for specific eukaryotic tissues, i.e. collagenase to break down collagen prior to cell lysis.

Most animal tissues can be processed fresh (unfrozen). When disrupting fresh tissue, the cells need to be sheared immediately at the time the GITC lysis solution is added if RNA isolation is desired. This can be done by dispensing the lysis solution in the Dounce or tube, adding the tissue and immediately sonicating or homogenizing. Hard tissues such as bone, teeth and some hard tumors may require milling. SPEX CertiPrep, Inc. of Metuchen, N.J. (732-549-7144) manufactures freezer mills that pulverize samples by shuttling an impactor back and forth magnetically at cryogenic temperatures.

Animal tissues that have been frozen after collection are disrupted by grinding in liquid nitrogen with a mortar and pestle. Grinding may be followed by thorough homogenization with a Dounce or mechanical homogenizer in a GITC lysis buffer.

Cultured cells are normally easy to disrupt. Cells grown in suspension are collected by centrifugation, washed and resuspended in a lysis solution (such as the GITC lysis solution). Lysis is made complete by immediate vortexing or vigorous pipetting of the solution.

Soft, fresh plant tissues from plants such as Nicotiana and Arabidopsis can often be disrupted by homogenization in lysis buffer alone. Other plant tissues, like pine needles, can be ground dry, without liquid nitrogen. Some hard, woody plant may require freezing and grinding in liquid nitrogen or milling. Plant cell suspension cultures and calli can be lysed by sonication in a lysis buffer for 0.5-2 minutes.

Yeast can be extremely difficult to disrupt because their cell walls may form capsules or nearly indestructible spores. There are several ways to approach yeast cell disruption. One method is mechanical disruption using a bead mill. Bead mills vigorously agitate a tube containing the sample, lysis buffer and small glass beads (0.5-1 mm). In a few minutes, cells are completely disrupted. Alternatively, yeast cell walls can be digested with zymolase, glucalase and/or lyticase to produce spheroplasts which are readily lysed by vortexing in a guanidinium-based lysis buffer. Some specialized isolation methods for yeast exist which use such methods as boiling SDS or boiling phenol treatment to insure complete cell lysis.

To disrupt filamentous fungi, scrape the mycelial mat into a cold mortar, add liquid nitrogen and grind to a fine powder with a pestle. The powder can then be thoroughly homogenized or sonicated in lysis buffer to completely solubilize.

Bacteria, like plants, are extremely diverse; therefore, conditions for breaking the cell walls and membrane vary. Bead milling will lyse most Gram positive and Gram negative bacteria, including mycobacteria. It can be performed by adding glass beads and lysis solution to a bacterial cell pellet and milling for a few minutes. It is possible to lyse some Gram negative bacteria by sonication in lysis solution alone.

Bacterial cell walls can be digested with lysozyme to form spheroplasts. Gram positive bacteria usually require more rigorous digestion (increased incubation time, increased incubation temperature, etc.) than Gram negative organisms. The spheroplasts are then easily lysed with vigorous vortexing or sonication in GITC lysis buffer.

In one aspect of the invention, methods are provided for differential isolation of cellular contents from interested cells in a sample with mixed cell population. Some embodiments of the invention employs differential lysis of cells to isolate contents of different cells (e.g., bacteria cells vs. animal cells) for further analyses such as gene expression monitoring or microorgnism genetic identification using oligonucleotide probe arrays.

In some embodiments, the methods include obtaining a sample comprising a first type of cells and a second type of cells, where the first type of cells is at least twice as susceptible to a lysis agent as the second type of cells; and applying the lysis agent to break the first type of cells. As used herein, the term lysis agent refers to any physical, biological, or chemical force that causes the break down of cell walls or cell membranes. Exemplary lysis agents include detegents, enzymes, sonication, heat, mechanic forces, etc.

In some embodiments, the first type of cells is at least 5 or 10 times more susceptible to the lysis agent than the second type of cells. In some embodiments, at least 60%, 80%, 90% or 99% of the second type of cells are removed by, for example, centrifugation.

The first type of cells can be animal cells and the second type of cells are bacteria cells. In some other embodiments, the first type of cells are animal cells and the second type of cells are plant cells or fungi cells. In such embodiments, the lysis agent can be a detergent or incubation with a detergent.

The method can also be used to separate bacterial cells from difference species. For example, the first type of cells can be gram negative bacteria and the second type of cells can be gram positive bacteria. The lysis agent can be a relatively mild lysosome digestion followed by a cell membrane lysis agent with the conditions that is sufficient for digesting gram negative bacteria and not sufficient for digesting gram positive bacteria.

In another exemplary embodiment, the first type of cells are yeast cells and the second type of cells are bacteria or plant cells. The lysis agent is a zymolase, glucalase or lyticase digestion followed by a cell membrane lysis agent.

In another aspect of the invention, methods are provided for detecting nucleic acids. The methods include the steps of obtaining a sample containing a first type of cells and a second type of cells, where the first type of cells is at least twice as susceptible to a lysis agent as the second type of cells; applying the lysis agent to break the first type of cells; removing at least 60% of the second type of cells to obtain an isolate; preparing a nucleic acid sample from the isolate.

In some embodiments, the first type of cells is at least 5 or 10 times more susceptible to the lysis agent than the second type of cells. In some embodiments, at least 80%, 90% or 99% of the second type of cells are removed.

The nucleic acid sample may be used to hybridize to a plurality of, at least 100, 1000, 10000, 300,000 different nucleic acid probes. The probes may be immobilized on a substrate which can be solid surface such a glass plate or slide, a gel matrix. In such embodiments, each of the different probes is immobilized on an addressable location. The probes may also be immobilized, for example, on beads or on optical fibers.

The first type of cells can be animal cells and the second type of cells are bacteria cells. In some other embodiments, the first type of cells are animal cells and the second type of cells are plant cells or fungi cells. In such embodiments, the lysis agent can be a detergent or incubation with a detergent.

The method can also be used to separate bacterial cells from difference species. For example, the first type of cells can be gram negative bacteria and the second type of cells can be gram positive bacteria. The lysis agent can be a relatively mild lysosome digestion followed by a cell membrane lysis agent with the conditions that is sufficient for digesting gram negative bacteria and not sufficient for digesting gram positive bacteria.

The nucleic acid sample can be used for gene expression monitoring. In such embodiments, the nucleic acid sample contains RNA transcripts from the first type of cells or nucleic acids derived from the RNA transcripts from the first type of cells and wherein the nucleic acid probes are for detecting gene expression.

In another exemplary embodiment, genomic DNA from the first type of cells or nucleic acids derived from the genomic DNA from the first type of cells are hybridized with the nucleic acid probes that are designed for identifying the species of the first type of cells according to the genomic DNA sequenceIn yet another aspect of the invention, methods for detecting nucleic acids from the second type of cells are provided. In some embodiments, the methods include obtaining a sample comprising a first type of cells and a second type of cells, where the first type of cells is at least twice as susceptible to a lysis agent as the second type of cells; applying the lysis agent to break the first type of cells; removing at least 80% of the cellular content of first type of cells; lysing second type of cells to obtain an isolate; and preparing a nucleic acid sample from the isolate.

In some embodiments, the first type of cells is at least 5 or 10 times more susceptible to the lysis agent than the second type of cells. In some embodiments, at least 90%, or 99% of the cellular content of the first type of cells is removed.

The first type of cells can be animal cells and the second type of cells are bacteria cells. In some other embodiments, the first type of cells are animal cells and the second type of cells are plant cells or fungi cells. In such embodiments, the lysis agent can be a detergent or incubation with a detergent.

The method can also be used to separate bacterial cells from difference species. For example, the first type of cells can be gram negative bacteria and the second type of cells can be gram positive bacteria. The lysis agent can be a relatively mild lysosome digestion followed by a cell membrane lysis agent with the conditions that is sufficien the second type of cells; applying the lysis agent to break the first type of cells; removing at least 60% of the second type of cells to obtain an isolate; preparing a nucleic acid sample from the isolate.

In some embodiments, the first type of cells is at least 5 or 10 times more susceptible to the lysis agent than the second type of cells. In some embodiments, at least 80%, 90% or 99% of the second type of cells are removed.

The nucleic acid sample may be used to hybridize to a plurality of, at least 100, 1000, 10000, 300,000 different nucleic acid probes. The probes may be immobilized on a substrate which can be solid surface such a glass plate or slide, a gel matrix. In such embodiments, each of the different probes is immobilized on an addressable location. The probes may also be immobilized, for example, on beads or on optical fibers.

The first type of cells can be animal cells and the second type of cells are bacteria cells. In some other embodiments, the first type of cells are animal cells and the second type of cells are plant cells or fungi cells. In such embodiments, the lysis agent can be a detergent or incubation with a detergent.

The method can also be used to separate bacterial cells from difference species. For example, the first type of cells can be gram negative bacteria and the second type of cells can be gram positive bacteria. The lysis agent can be a relatively mild lysosome digestion followed by a cell membrane lysis agent with the conditions that is sufficient for digesting gram negative bacteria and not sufficient for digesting gram positive bacteria.

The nucleic acid sample can be used for gene expression monitoring. In such embodiments, the nucleic acid sample contains RNA transcripts from the second type of cells or nucleic acids derived from the RNA transcripts from the second type of cells and wherein the nucleic acid probes are for detecting gene expression.

In another exemplary embodiment, genomic DNA from the second type of cells or nucleic acids derived from the genomic DNA from the first type of cells are hybridized with the nucleic acid probes that are designed for identifying the species of the second type of cells according to the genomic DNA sequence.

While the embodiments of the invention are primarily described in terms of separating the cellular content of one type of cells from another type, the embodiments of the invention are not limited to cell mixtures that contain two types of cells, rather, the embodiments may be useful for processing cell mixtures containing multiple type of cells. For example, a mixture containing animal, yeast and bacterial cells may be used to isolate cellular content of any of the cells. In one embodiment, the cellular content of animal cells is obtained by lysing the mixture with a detergent. The intact cells are separated by centrifugation to obtain a second mixture containing yeast and bacteria cells. The second mixture may be subject to enzymatic digestion to break the cell walls of either yeast or bacterial cells, depending upon the enzyme used and the condition of digestion. The cells with cell walls removed can be easily and differentially lysed while the cells with cell wall remain intact.

As used herein, a nucleic acid derived from a RNA transcript refers to a nucleic acid for whose synthesis the RNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample.

Similarly, a nucleic acid derived from a genome DNA refers to a nucleic acid for whose synthesis the RNA transcript or a subsequence thereof has ultimately served as a template.

Transcripts, as used herein, may include, but not limited to pre-mRNA nascent transcript(s), transcript processing intermediates, mature mRNA(s) and degradation products. It is not necessary to monitor all types of transcripts to practice this invention. For example, one may choose to practice the invention to measure the mature mRNA levels only.

Biological samples may be of any biological tissue or fluid or cells. Frequently the sample will be a “clinical sample” which is a sample derived from a patient. Clinical samples provide a rich source of information regarding the various states of genetic network or gene expression. Some embodiments of the invention are employed to detect mutations and to identify the function of mutations. Such embodiments have extensive applications in clinical diagnostics and clinical studies. Typical clinical samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

Clinical samples frequently contain animal and bacterial cells. The bacterial or animal cell contents may be isolated using differential lysis of cell walls and cell membrane.

Methods of isolating total RNA and mRNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993)).

In a preferred embodiment, the total RNA is isolated from a given sample using, for example, an acid guanidinium-phenol-chloroform extraction method and polyA+ mRNA is isolated by oligo (dT) column chromatography or by using (dT) magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Most of eukaroytic mRNA have 3″ poly (A) tails, some of eukaroytic and all of prokaroytic mRNA do not contain 3″ poly (A) tails. It is often desirable to isolate mRNAs from RNA samples.

In one particularly preferred embodiment, total RNA is isolated from mammalian cells using RNeasy Total RNA isolation kit (QIAGEN). If mammalian tissue is used as the source of RNA, a commercial reagent such as TRIzol Reagent (Life Technologies). A second cleanup after the ethanol precipitation step in the TRIzol extraction using Rneasy total RNA isolation kit may be beneficial.

Hot phenol protocol described by Schmitt, et al., (1990) Nucleic Acid Res., 18:3091-3092 is useful for isolating total RNA for yeast cells.

Good quality mRNA may be obtained by, for example, first isolating total RNA and then isolating the mRNA from the total RNA using Oligotex mRNA kit (QIAGEN).

Total RNA from prokaryotes, such as E. coli. Cells, may be obtained by following the protocol for MasterPure complete DNA/RNA purification kit from Epicentre Technologies (Madison, Wis.).

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids to achieve quantitative amplification.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid.

Other suitable amplification methods include, but are not limited to polymerase chain reaction (PCR) (Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

Cell lysates or tissue homogenates often contain a number of inhibitors of polymerase activity. Therefore, RT-PCR typically incorporates preliminary steps to isolate total RNA or mRNA for subsequent use as an amplification template. One tube mRNA capture method may be used to prepare poly(A)+ RNA samples suitable for immediate RT-PCR in the same tube (Boehringer Mannheim). The captured mRNA can be directly subjected to RT-PCR by adding a reverse transcription mix and, subsequently, a PCR mix.

In a particularly preferred embodiment, the sample mRNA is reverse transcribed with a reverse transcriptase and a primer consisting of oligo dT, random hexamer, random nanomer or other primers and to provide a single stranded DNA template. The reverse transcription reactions are preferred performed in a condition that suppresses the hairpin formation to reduce second strand cDNA synthesis. For example, actinomycin D (Actinomycin D with mannitol (Sigma) was dissolved in water to a stock concentration of 1 mg/ml.) may be added before the reverse transcription reaction is initiated. One of skill in the art would appreciate that the scope of the invention is not limited to the particular concentration described herein. It is well within the skill of one of ordinary skills in the art to optimize assays by varying the concentration of reagents according to the need to particular experiment purpose and experimental conditions.

Before hybridization, the resulting cRNA or cDNA may be fragmented. One preferred method for fragmentation employs Rnase free RNA fragmentation buffer (200 mM tris-acetate, pH 8.1, 500 mM potassium acetate, 150 mM magnesium acetate). Approximately 20 μg of cRNA is mixed with 8 μL of the fragmentation buffer. Rnase free water is added to make the volume to 40 μL. The mixture may be incubated at 94° C. for 35 minutes and chilled in ice.

The biological sample should contain nucleic acids that reflects the level of at least some of the transcripts present in the cell, tissue or organ of the species of interest. In some embodiments, the biological sample may be prepared from cell, tissue or organs of a particular status. For example, a total RNA preparation from the pituitary of a dog when the dog is pregnant. In another example, samples may be prepared from E. Coli cells after the cells are treated with IPTG. Because certain genes may only be expressed under certain conditions, biological samples derived under various conditions may be needed to observe all transcripts. In some instance, the transcriptional annotation may be specific for a particular physiological, pharmacological or toxicological condition. For example, certain regions of a gene may only be transcribed under specific physiological conditions. Transcript annotation obtained using biological samples from the specific physiological conditions may not be applicable to other physiological conditions.

### Hybridization and Washing

Nucleic acid hybridization simply involves contacting a probe and target nucleic acid under conditions where the probe and its complementary target can form stable hybrid duplexes through complementary base pairing.

It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency in this case in 6×SSPE-T at 37 C. (0.005% Triton X-100) to ensure hybridization and then subsequent washes are performed at higher stringency (e.g., 1×SSPE-T at 37 C.) to eliminate mismatched hybrid duplexes. Successive washes may be performed at increasingly higher stringency (e.g., down to as low as 0.25×SSPE-T at 37 C. to 50 C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Altering the thermal stability (Tm) of the duplex formed between the target and the probe using, e.g., known oligonucleotide analogues allows for optimization of duplex stability and mismatch discrimination. One useful aspect of altering the Tm arises from the fact that adenine-thymine (A-T) duplexes have a lower Tm than guanine-cytosine (G-C) duplexes, due in part to the fact that the A-T duplexes have 2 hydrogen bonds per base-pair, while the G-C duplexes have 3 hydrogen bonds per base pair. In heterogeneous oligonucleotide arrays in which there is a non-uniform distribution of bases, it is not generally possible to optimize hybridization for each oligonucleotide probe simultaneously. Thus, in some embodiments, it is desirable to selectively destabilize G-C duplexes and/or to increase the stability of A-T duplexes. This can be accomplished, e.g., by substituting guanine residues in the probes of an array which form G-C duplexes with hypoxanthine, or by substituting adenine residues in probes which form A-T duplexes with 2,6 diaminopurine or by using the salt tetramethyl ammonium chloride (TMACl) in place of NaCl.

Methods of optimizing hybridization conditions are well known to those of skill in the art (see, e.g., Laboratory Techniques in Biochemistry and Molecular Biology, Vol. 24: Hybridization With Nucleic Acid Probes, P. Tijssen, ed. Elsevier, N.Y., (1993)).

### Signal Detection

In a preferred embodiment, the hybridized nucleic acids are detected by detecting one or more labels attached to the sample nucleic acids. See, e.g., U.S. Pat. No. 6,225, 625, which is incorporated herein by reference. The labels may be incorporated by any of a number of means well known to those of skill in the art. However, in a preferred embodiment, the label is simultaneously incorporated during the amplification step in the preparation of the sample nucleic acids. Thus, for example, polymerase chain reaction (PCR) with labeled primers or labeled nucleotides will provide a labeled amplification product. In a preferred embodiment, transcription amplification, as described above, using a labeled nucleotide (e.g. fluorescein-labeled UTP and/or CTP) incorporates a label into the transcribed nucleic acids. Alternatively, cDNAs synthesized using a RNA sample as a template, cRNAs are synthesized using the cDNAs as templates using in vitro transcription (IVT). A biotin label may be incorporated during the IVT reaction (Enzo Bioarray high yield labeling kit).

Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 1251, 35S, 14C, or 32P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; 4,366,241, and 5,800,992.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label. One particularly preferred method uses colloidal gold label that can be detected by measuring scattered light.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an aviden-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are preferred and easily added during an in vitro transcription reaction. In a preferred embodiment, fluorescein labeled UTP and CTP are incorporated into the RNA produced in an in vitro transcription reaction as described above.

Means of detecting labeled target (sample) nucleic acids hybridized to the probes of the high density array are known to those of skill in the art. Thus, for example, where a colorimetric label is used, simple visualization of the label is sufficient. Where a radioactive labeled probe is used, detection of the radiation (e.g. with photographic film or a solid state detector) is sufficient.

In a preferred embodiment, however, the target nucleic acids are labeled with a fluorescent label and the localization of the label on the probe array is accomplished with fluorescent microscopy. The hybridized array is excited with a light source at the excitation wavelength of the particular fluorescent label and the resulting fluorescence at the emission wavelength is detected. In a particularly preferred embodiment, the excitation light source is a laser appropriate for the excitation of the fluorescent label.

The confocal microscope may be automated with a computer-controlled stage to automatically scan the entire high density array. Similarly, the microscope may be equipped with a phototransducer (e.g., a photomultiplier, a solid state array, a CCD camera, etc.) attached to an automated data acquisition system to automatically record the fluorescence signal produced by hybridization to each oligonucleotide probe on the array. Such automated systems are described at length in U.S. Pat. No. 5,143,854, PCT Application 20 92/10092, and U.S. application Ser. No. 08/195,889 filed on Feb. 10, 1994. Use of laser illumination in conjunction with automated confocal microscopy for signal detection permits detection at a resolution of better than about 100 μm, more preferably better than about 50 μm, and most preferably better than about 25 μm.

One of skill in the art will appreciate that methods for evaluating the hybridization results vary with the nature of the specific probe nucleic acids used as well as the controls provided. In the simplest embodiment, simple quantification of the fluorescence intensity for each probe is determined. This is accomplished simply by measuring probe signal strength at each location (representing a different probe) on the high density array (e.g., where the label is a fluorescent label, detection of the amount of florescence (intensity) produced by a fixed excitation illumination at each location on the array). Comparison of the absolute intensities of an array hybridized to nucleic acids from a “test” sample with intensities produced by a “control” sample provides a measure of the relative expression of the nucleic acids that hybridize to each of the probes.

One of skill in the art, however, will appreciate that hybridization signals will vary in strength with efficiency of hybridization, the amount of label on the sample nucleic acid and the amount of the particular nucleic acid in the sample. Typically nucleic acids present at very low levels (e.g., <1 pM) will show a very weak signal. At some low level of concentration, the signal becomes virtually indistinguishable from the background. In evaluating the hybridization data, a threshold intensity value may be selected below which a signal is not counted as being essentially indistinguishable from the background.

Suitable scanners, computer software for controlling the scanners and computer software for data management and analysis are available from commercial sources, such as Affymetrix, Inc., Santa Clara, Calif.

### EXAMPLES

This example shows the isolation of animal RNA from a cell mixture containing cultured animal cells and E. coli.

Materials and Methods HUVEC cells (Biowhittaker, Rockland, Me.) were grown to ˜99% confluency. The starting seeding density is 2500 cells/cm² in a 25 ml flask for 5 days at 37° C. and 5% CO₂ in HUVEC cell media (Biowhittaker, Rockland, Me.) as recommended in the media kit protocol provided with the cells. One hour before microbial infection with E. coli, media is removed from the cells and replaced with prewarmed antibiotic free media for HUVEC cells (Biowhittaker, Rockland, Me.). The E. coli cells are added to the HUVEC cells at a 100:1 ratio (MOI 100). The infected culture is incubated for 30 minutes in 5% CO₂ at 37° C.

The cells are removed by scraping of the flask and pipetted into a centrifuge tube. A final concentration of 0.2% Triton X-100 is added to the HUVEC/E. coli cell mixture to selectively lyse the HUVEC cells, inverted 4-5 times and spun down immediately for 2 minutes. At this step a transcriptional inhibitor could be added to freeze the expression profile.

The pellet contains the E. coli RNA and is isolated using the Epicentre RNA isolation protocol. The supernatant contains the human RNA and is further isolated using the Rneasy protocol after RLN lysis (Qiagen, Valencia, Calif.). After the Triton x-100 addition, the E. coli pellet will have a viscous look and part of the pellet may be detached from its core. To aid in isolating more RNA, the detached portion can be lysed and purified along with the rest of the pellet.

Isolated total bacterial RNA was used to synthesize cDNA using the standard cDNA synthesis protocol and hybridized to the E. coli genome array. Depending on the infection ratio and amounts of starting cells an improved cDNA hybridization protocol was developed. This hybridization protocol can detect similar number of expressed genes with as little as 100 ng cDNA. The hybridization solution was x μl of target (100 ng fragmented, labeled cDNA), 2.2 μl HS-DNA, 2.2 μl Bio-948 oligo control, 4.4 μl 1% Triton, 132 μl 5M TMAC, 18 μl 12×MES, and add water to 220 μl. Standard temperature, hybridization, and stain protocols were used.

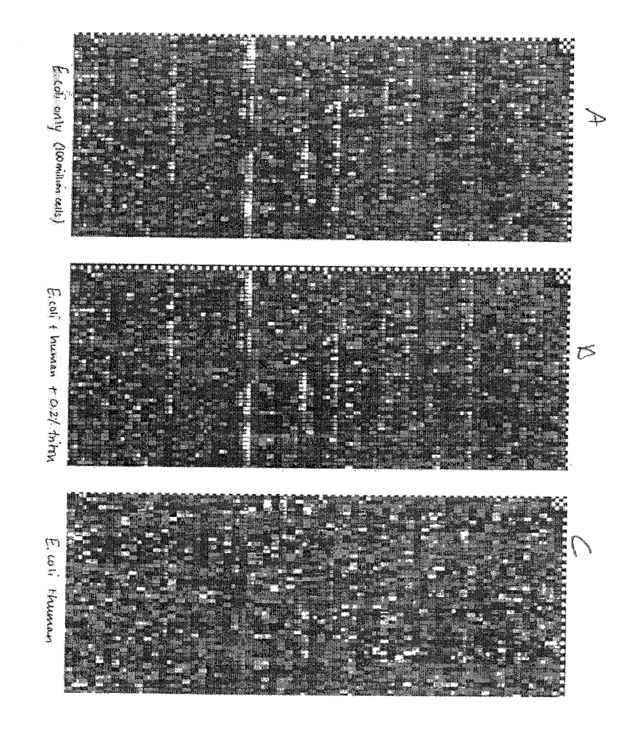

Result FIGS. 1A, 1B and 1C shows the hybridization pattern of RNAs from various sources. FIG. 1A shows the hybridization of RNA from 100 million E. coli. cells. FIG. 1B shows the hybridization of RNA from a mixture of E. Coli and Human cells (100:1). The RNA was isolated by first lysing human cells with 0.2% Triton. The hybridization patterns shown in FIGS. 1A and 1B are almost identical, which indicates that the human cells did not become a source of RNA. In contrast, when RNA were isolated from a mixture (100:1) E. coli. and human cells without Triton lysis, the hybridization pattern is very different (FIG. 1C), indicating human RNAs became a significant source of the target RNA.

It is to be understood that the above descripis intended to be illustrative and not restrictive. Many variations of the invention will be apparent to those of skill in the art upon reviewing the above description. By way of example, the invention has been described primarily with reference to the use of a high density oligonucleotide array, but it will be readily recognized by those of skill in the art that other nucleic acid arrays. The scope of the invenshould be deterwith reference to the appended claims, along with the full scope of equivalents to which such claims are entitled. All cited references, including patent and non-patent literature, are incorporated herewith by reference in their entireties for all purposes.

1. A method for sample preparation comprising:
obtaining a sample comprising a first type of cells and a second type of cells, wherein the first type of cells is at least twice as susceptible to a lysis agent as the second type of cells; and
applying the lysis agent to break the first type of cells.

2. The method of claim 1 wherein the first type of cells is at least 5 times more susceptible to the lysis agent than the second type of cells.

3. The method of claim 2 wherein the first type of cells is at least 10 times more susceptible to the lysis agent than the second type of cells.

4. The method of claim 2 further comprising removing at least 60% of the second type of cells.

5. The method of claim 4 further comprising removing at least 80% of the second type of cells.

6. The method of claim 5 further comprising removing at least 90% of the second type of cells.

7. The method of claim 6 further comprising removing at least 99% of the second type of cells.

8. The method of claim 2 further comprising removing at least 90% the cellular content of first type of cells to obtain an isolation comprising second type of cells.

9. The method of claim 8 further comprising removing at least 99% of the cellular content of the first type of cells to obtain an isolation comprising secind type of cells.

10. The method of claim 6, 7, 8, or 9 wherein the first type of cells are animal cells and the second type of cells are bacteria cells.

11. The method of claim 10 wherein the lysis agent is a detergent.

12. The method of claim 6, 7, 8, or 9 wherein the first type of cells are animal cells and the second type of cells are plant cells.

13. The method of claim 6, 7, 8, or 9 wherein the first type of cells are animal cells and the second type of cells are fungi cells.

14. The method of claim 6, 7, 8, or 9 wherein the first type of cells are gram negative bacteria and the second type of cells are gram positive bacteria.

15. The method of claim 14 wherein the lysing agent is a relatively mild lysosome digestion followed by a cell membrane lysis agent with the conditions that is sufficient for digesting gram negative bacteria and not sufficient for digesting gram positive bacteria.

16. The method of claim 6, 7, 8 or 9 wherein the first type of cells are yeast cells and the second type of cells are bacteria or plant cells.

17. The method of claim 16 wherein the lysis agent is a zymolase, glucalase or lyticase digestion followed by a cell membrane lysis agent.

18. A method for detecting nucleic acids comprising
obtaining a sample comprising a first type of cells and a second type of cells, wherein the first type of cells is at least twice as susceptible to a lysis agent as the second type of cells;
applying the lysis agent to break the first type of cells;
removing at least 60% of the second type of cells to obtain an isolate;
preparing a nucleic acid sample from the isolate.

19. The method of claim 18 wherein the first type of cells is at least 5 times more susceptible to the lysis agent than the second type of cells.

20. The method of claim 19 wherein the first type of cells is at least 10 times more susceptible to the lysis agent than the second type of cells.

21. The method of claim 19 wherein the removing comprises removing at least 80% of the second type of cells.

22. The method of claim 21 wherein the removing comprises removing at least 90% of the second type of cells.

23. The method of claim 22 wherein the removing comprises removing at least 99% of the second type of cells.

24. The method of claim 23 further comprising hybridizing the nucleic acid sample to a plurality of different nucleic acid probes.

25. The method of claim 23 wherein the plurality of different nucleic acid probes comprises at least 100 probes.

26. The method of claim 25 wherein the plurality of different nucleic acid probes comprises at least 1000 probes.

27. The method of claim 26 wherein the pluarlity of different nucleic acid probes comprise at least 5000 probes.

28. The method of claim 27 wherein the plurality of nucleic acid probes are immobilized on a substrate, each of the different probes is immobilized on a different location.

29. The method of claim 28 wherein the substrate is a solid surface.

30. The method of claim 28 wherein the substrate is a matrix.

31. The method of claim 27 wherein each of the different probes is immobilized on a bead.

32. The method of claim 27 wherein each of the different probes is immobilized on an optical fiber.

33. The method of claim 29 wherein the first type of cells are animal cells and the second type of cells are bacteria cells.

34. The method of claim 33 wherein the lysis agent is a detergent.

35. The method of claim 29 wherein the first type of cells are animal cells and the second type of cells are plant cells.

36. The method of claim 29 wherein the first type of cells are animal cells and the second type of cells are fungi cells.

37. The method of claim 29 wherein the first type of cells are gram negative bacteria and the second type of cells are gram positive bacteria.

38. The method of claim 37 wherein the lysing agent is a relatively mild lysosome digestion followed by a cell membrane lysis agent with the conditions that is sufficient for digesting gram negative bacteria and not sufficient for digesting gram positive bacteria.

39. The method of claim 29 wherein the first type of cells are yeast cells and the second type of cells are bacteria or plant cells.

40. The method of claim 39 wherein the lysis agent is a zymolase, glucalase or lyticase digestion followed by a cell membrane lysis agent.

41. The method of claim 29 wherein the nucleic acid sample comprising RNA transcripts from the first type of cells or nucleic acids derived from the RNA transcripts from the first type of cells and wherein the nucleic acid probes are for detecting gene expression.

42. The method of claim 29 wherein the nucleic acid sample comprising genomic DNA from the first type of cells or nucleic acids derived from the genomic DNA from the first type of cells and wherein the nucleic acid probes are for identifying the species of the first type of cells according to the genomic DNA sequence.

43. A method for detecting nucleic acids comprising
obtaining a sample comprising a first type of cells and a second type of cells, wherein the first type of cells is at least twice at susceptible to a lysis agent as the second type of cells;
applying the lysis agent to break the first type of cells;
removing at least 80% of the cellular content of first type of cells;
lysing second type of cells to obtain an isolate; and
preparing a nucleic acid sample from the isolate.

44. The method of claim 43 wherein the first type of cells is at least 5 times more susceptible to the lysis agent than the second type of cells.

45. The method of claim 44 wherein the first type of cells is at least 10 times more susceptible to the lysis agent than the second type of cells.

46. The method of claim 45 wherein the removing comprises removing at least 90% of the cellular content of the first type of cells.

47. The method of claim 46 wherein the removing comprises removing at least 99% of the cellular content of the first type of cells.

48. The method of claim 47 further comprising hybridizing the nucleic acid sample to a plurality of different nucleic acid probes.

49. The method of claim 49 wherein the plurality of different nucleic acid probes comprises at least 100 probes.

50. The method of claim 49 wherein the plurality of different nucleic acid probes comprises at least 1000 probes.

51. The method of claim 50 wherein the pluarlity of different nucleic acid probes comprise at least 5000 probes.

52. The method of claim 51 wherein the plurality of nucleic acid probes are immobilized on a substrate, each of the different probes is immobilized on a different location.

53. The method of claim 52 wherein the substrate is a solid surface.

54. The method of claim 52 wherein the substrate is a matrix.

55. The method of claim 51 wherein each of the different probes is immobilized on a bead.

56. The method of claim 51 wherein each of the different probes is immobilized on an optical fiber.

57. The method of claim 51 wherein the first type of cells are animal cells and the second type of cells are bacteria cells.

58. The method of claim 57 wherein the lysis agent is a detergent.

59. The method of claim 51 wherein the first type of cells are animal cells and the second type of cells are plant cells.

60. The method of claim 51 wherein the first type of cells are animal cells and the second type of cells are fungi cells.

61. The method of claim 51 wherein the first type of cells are gram negative bacteria and the second type of cells are gram positive bacteria.

62. The method of claim 61 wherein the lysing agent is a relatively mild lysosome digestion followed by a cell membrane lysis agent with the conditions that is sufficient for digesting gram negative bacteria and not sufficient for digesting gram positive bacteria.

63. The method of claim 51 wherein the first type of cells are yeast cells and the second type of cells are bacteria or plant cells.

64. The method of claim 63 wherein the lysis agent is a zymolase, glucalase or lyticase digestion followed by a cell membrane lysis agent.

65. The method of claim 51 wherein the nucleic acid sample comprising RNA transcripts from the second type of cells or nucleic acids derived from the RNA transcripts from the second type of cells and wherein the nucleic acid probes are for detecting gene expression.

66. The method of claim 51 wherein the nucleic acid sample comprising genomic DNA from the second type of cells or nucleic acids derived from the genomic DNA from the second type of cells and wherein the nucleic acid probes are for identifying the species of the first type of cells according to the genomic DNA sequences
